(19) United States
(12) Patent Application Publication
(10) Pub. No.: US 2023/0333121 A1
(43) Pub. Date: Oct. 19, 2023

(54) METHODS AND COMPOSITIONS RELATING TO BIOMARKERS FOR NEURODEGENERATIVE DISEASES

(71) Applicant: ANACALYPSIS THERAPEUTICS, IKE, Moschato (GR)

(72) Inventor: Hardy J. RIDEOUT, Athens (GR)

(21) Appl. No.: 18/308,318

(22) Filed: Apr. 27, 2023

Related U.S. Application Data
(63) Continuation of application No. PCT/IB2021/000754, filed on Oct. 29, 2021.

(30) Foreign Application Priority Data
Oct. 30, 2020 (GR) 20200100660

(51) Int. Cl. G01N 33/68

(52) U.S. Cl. CPC G01N 33/6896; G01N 2333/91205; G01N 2440/14; G01N 2800/50; G01N 2800/2835; G01N 2800/52; G01N 2800/56

(57) ABSTRACT

Provided herein are methods for making a clinical decision in an individual having or suspected of having a neurodegenerative disease or disorder by determining a level, post-translational modification, or both of a plurality of biomarkers.

### CROSS-REFERENCE

This application is a continuation of International Patent Application No. PCT/IB2021/000754, filed Oct. 29, 2021, which claims the benefit of Greek Application No. 20200100660 filed on Oct. 30, 2020, which is incorporated by reference in its entirety.

### BRIEF SUMMARY

Provided herein are methods for making a clinical decision in an individual having, suspected of having, or at risk of progressing to a neurodegenerative disease or disorder, comprising: a) obtaining a biological sample from the individual having, suspected of having, or at risk of progressing to the neurodegenerative disease or disorder; b) performing an assay on the biological sample to determine a level, post-translational modification, or both of LRRK2, alpha-synuclein, Rab8a, Rab10, Rab12, and Rab29; and c) making the clinical decision based on the level, post-translational modification, or both of LRRK2, alpha-synuclein, Rab8a, Rab10, Rab12, and Rab29. Provided herein are methods for making a clinical decision in an individual having, suspected of having, or at risk of progressing to a neurodegenerative disease or disorder, comprising: a) performing an assay on a biological sample obtained from the individual having, suspected of having, or at risk of progressing to the neurodegenerative disease or disorder to determine a level, post-translational modification, or both of LRRK2, alpha-synuclein, Rab8a, Rab10, Rab12, and Rab29; and b) making the clinical decision based on the level, post-translational modification, or both of LRRK2, alpha-synuclein, Rab8a, Rab10, Rab12, and Rab29. Further provided herein are methods, wherein the clinical decision is determining a prognosis for the individual. Further provided herein are methods, wherein the clinical decision is determining eligibility of the individual for a clinical trial. Further provided herein are methods, wherein the clinical decision is designing a clinical trial. Further provided herein are methods, wherein the clinical trial is selected from the group consisting of pre-clinical trial, Phase I clinical trial, Phase II clinical trial, and Phase III clinical trial. Further provided herein are methods, wherein the clinical decision is related to drug screening. Further provided herein are methods, wherein the clinical decision is related to dose finding. Further provided herein are methods, wherein the clinical decision is determining efficacy of a drug in a clinical trial. Further provided herein are methods, wherein the clinical decision is measuring target engagement of an investigational compound. Further provided herein are methods, wherein the clinical decision is determining exclusion criteria, inclusion criteria, or both for a clinical trial of an investigational compound. Further provided herein are methods, wherein the clinical decision comprises determining a risk of the individual in developing the neurodegenerative disease or disorder. Further provided herein are methods, wherein the clinical decision comprises determining a stage of the neurodegenerative disease or disorder in the individual. Further provided herein are methods, wherein the clinical decision comprises determining a severity of the neurodegenerative disease or disorder in the individual. Further provided herein are methods, wherein the clinical decision comprises determining a neurodegenerative disease or disorder therapy. Further provided herein are methods, wherein the clinical decision comprises determining an endpoint for the neurodegenerative disease or disorder therapy. Further provided herein are methods, further comprising determining an individual's responsiveness to the neurodegenerative disease or disorder therapy based on the level, post-translational modification, or both of LRRK2, alpha-synuclein, Rab8a, Rab10, Rab12, and Rab29. Further provided herein are methods, wherein determining the individual's responsiveness to the neurodegenerative disease or disorder therapy comprises determining a likelihood of an adverse response to the neurodegenerative disease or disorder therapy. Further provided herein are methods, wherein determining the individual's responsiveness to the neurodegenerative disease or disorder therapy comprises determining a likelihood of a beneficial response to the neurodegenerative disease or disorder therapy. Further provided herein are methods, further comprising modifying the neurodegenerative disease or disorder therapy based on the level, post-translational modification, or both of LRRK2, alpha-synuclein, Rab8a, Rab10, Rab12, and Rab29. Further provided herein are methods, further comprising repeating steps (a) to (c) with a biological sample from the individual after the individual has received the neurodegenerative disease or disorder therapy and monitoring effectiveness of the neurodegenerative disease or disorder therapy by monitoring for a change in the level, post-translational modification, or both of LRRK2, alpha-synuclein, Rab8a, Rab10, Rab12, and Rab29. Further provided herein are methods, wherein the neurodegenerative disease or disorder therapy is selected from the group consisting of a small molecule, an immunotherapy, a gene therapy, a non-medication based therapy, an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, and combinations thereof. Further provided herein are methods, wherein the assay measures total protein, phosphorylated protein, or aggregated protein. Further provided herein are methods, wherein the aggregated protein is selected from the group consisting of amorphous aggregates, oligomers, fibrils, and combinations thereof. Further provided herein are methods, wherein the level is a concentration. Further provided herein are methods, wherein the level is a ratio of phosphorylation to total protein. Further provided herein are methods, wherein the LRRK2 is total LRRK2. Further provided herein are methods, wherein the LRRK2 is pS935-LRRK2. Further provided herein are methods, wherein the LRRK2 is pS1292-LRRK2. Further provided herein are methods, wherein the alpha-synuclein is total alpha-synuclein. Further provided herein are methods, wherein the alpha-synuclein is pS129 alpha-synuclein. Further provided herein are methods, wherein the alpha-synuclein is aggregated alpha-synuclein. Further provided herein are methods, wherein the Rab8a is total Rab8a. Further provided herein are methods, wherein the Rab8a is pT72-Rab8a. Further provided herein are methods, wherein the Rab10 is total Rab10. Further provided herein are methods, wherein the Rab10 is pT73-Rab10. Further provided herein are methods, wherein the Rab12 is total Rab12. Further provided herein are methods, wherein the Rab12 is pS106-Rab12. Further provided herein are methods, wherein the Rab29 is total Rab29. Further provided herein are methods, wherein the Rab29 is pT71-Rab29. Further provided herein are methods, wherein step b) comprises determining a level of at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein step b) comprises determining a level of at least five proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein step b) comprises determining a level of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the assay is an immunoassay. Further provided herein are methods, wherein the assay is selected from the group consisting of a Western blot, Dot blot, enzyme-linked immunosorbent assays (ELISA), chemiluminescence, absorbance, and chromatography. Further provided herein are methods, wherein the assay is enzyme-linked immunosorbent assays (ELISA). Further provided herein are methods, wherein the ELISA comprises antibodies to at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the ELISA comprises antibodies to at least five proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the ELISA comprises antibodies to total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the ELISA comprises antibodies to total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 in a single well. Further provided herein are methods, wherein the biological sample is selected from the group consisting of whole blood, specific blood cell subtypes, plasma, serum, urine, cerebrospinal fluid, tears, saliva, skin, and exosomes. Further provided herein are methods, wherein the specific blood cell-subtypes is selected from the group consisting of peripheral blood mononuclear cells (PBMCs), neutrophils, lymphocytes, monocytes, eosinophils, basophils, and erythrocytes. Further provided herein are methods, wherein the neurodegenerative disease or disorder is selected from the group consisting of Parkinson's disease, multiple system atrophy (MSA); amyotrophic lateral sclerosis (ALS), progressive supranuclear palsy (PSP), corticobasal syndrome, Alzheimer's disease, frontotemporal dementia, multiple sclerosis (MS), corticobasal degeneration, motor neuron disease, spinocerebellar ataxia (SCA), spinal muscular atrophy (SMA), Huntington's disease (HD), Lewy body disease, Friedrich's ataxia, and synucleinopathies. Further provided herein are methods, wherein the neurodegenerative disease or disorder is Parkinson's disease. Further provided herein are methods, wherein the Parkinson's disease is familial Parkinson's disease. Further provided herein are methods, wherein the Parkinson's disease is sporadic Parkinson's disease.

Provided herein are methods comprising: a) obtaining a biological sample from an individual having, suspected of having, or at risk of progressing to a neurodegenerative disease or disorder; and b) performing an assay on the biological sample to determine a level, post-translational modification, or both of LRRK2, alpha-synuclein, Rab8a, Rab10, Rab12, and Rab29. Further provided herein are methods, wherein the assay measures total protein, phosphorylated protein, or aggregated protein. Further provided herein are methods, wherein the aggregated protein is selected from the group consisting of amorphous aggregates, dimers, oligomers, fibrils, and combinations thereof. Further provided herein are methods, wherein the level is a concentration. Further provided herein are methods, wherein the level is a ratio of phosphorylation to total protein. Further provided herein are methods, wherein the LRRK2 is total LRRK2. Further provided herein are methods, wherein the LRRK2 is pS935-LRRK2. Further provided herein are methods, wherein the LRRK2 is pS1292-LRRK2. Further provided herein are methods, wherein the alpha-synuclein is total alpha-synuclein. Further provided herein are methods, wherein the alpha-synuclein is pS129 alpha-synuclein. Further provided herein are methods, wherein the alpha-synuclein is aggregated alpha-synuclein. Further provided herein are methods, wherein the Rab8a is total Rab8a. Further provided herein are methods, wherein the Rab8a is pT72-Rab8a. Further provided herein are methods, wherein the Rab10 is total Rab10. Further provided herein are methods, wherein the Rab10 is pT73-Rab10 Further provided herein are methods, wherein the Rab12 is total Rab12. Further provided herein are methods, wherein the Rab12 is pS106-Rab12 Further provided herein are methods, wherein the Rab29 is total Rab29. Further provided herein are methods, wherein the Rab29 is pT71-Rab29. Further provided herein are methods, wherein step b) comprises determining a level of at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein step b) comprises determining a level of at least five proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein step b) comprises determining a level of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the assay is an immunoassay. Further provided herein are methods, wherein the assay is selected from the group consisting of a Western blot, Dot blot, enzyme-linked immunosorbent assays (ELISA), chemiluminescence, absorbance, and chromatography. Further provided herein are methods, wherein the assay is enzyme-linked immunosorbent assays (ELISA). Further provided herein are methods, wherein the ELISA comprises antibodies to at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the ELISA comprises antibodies to at least five proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the ELISA comprises antibodies to total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. Further provided herein are methods, wherein the ELISA comprises antibodies to total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 in a single well. Further provided herein are methods, wherein the biological sample is selected from the group consisting of whole blood, specific blood cell subtypes, plasma, serum, urine, cerebrospinal fluid, tears, saliva, skin, and exosomes. Further provided herein are methods, wherein the specific blood cell-subtypes is selected from the group consisting of peripheral blood mononuclear cells (PBMCs), neutrophils, lymphocytes, monocytes, eosinophils, basophils, and erythrocytes. Further provided herein are methods, wherein the neurodegenerative disease or disorder is selected from the group consisting of Parkinson's disease, multiple system atrophy (MSA); amyotrophic lateral sclerosis (ALS), progressive supranuclear palsy (PSP), corticobasal syndrome, Alzheimer's disease, frontotemporal dementia, multiple sclerosis (MS), corticobasal degeneration, motor neuron disease, spinocerebellar ataxia (SCA), spinal muscular atrophy (SMA), Huntington's disease (HD), Lewy body disease, Friedrich's ataxia, and synucleinopathies. Further provided herein are methods, wherein the neurodegenerative disease or disorder is Parkinson's disease. Further provided herein are methods, wherein the Parkinson's disease is familial Parkinson's disease. Further provided herein are methods, wherein the Parkinson's disease is sporadic Parkinson's disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a schema of the methods described herein.

FIGS. 2A-2F depict LRRK2 in vitro kinase activity in peripheral blood mononuclear cell (PBMC) extracts.

FIGS. 3A-3G depict pS935-LRRK2 ELISA in cell lines and human PBMCs.

FIG. 4 depicts Western blots of phosphorylation of Rab10 at Thr73 in extracts of PBMCs obtained from healthy control subjects, or subjects with idiopathic Parkinson's disease (PD) or carriers of the G2019S LRRK2 mutation, with and without PD (top panel) and total Rab10 (bottom panel).

FIGS. 5A-5B depict quantification of band intensity of Western immunoblots from PBMC extracts.

FIG. 6 depicts a Western immunoblot performed on PBMC extracts.

FIG. 7A depicts quantification of total LRRK2 at various dilution factors (1:27, 1:9, and 1:3).

FIG. 7B depicts quantification of pS935-LRRK2 at various dilution factors (1:27, 1:9, and 1:3).

FIG. 7C depicts quantification of levels of pS935-LRRK2 at the various CSF sample conditions.

FIG. 8 depicts pS935-LRRK2 levels following MLi-2 treatment.

FIG. 9 depicts data comparing two diluents in the detection of over-expressed WT human LRRK2.

### DETAILED DESCRIPTION

Neurodegenerative diseases and disorders comprise a heterogenous group of diseases and disorders that are characterized by progressive degeneration of the structure and function of the central nervous system or peripheral nervous system. Effective treatment and clinical decision making requires accurate detection of levels and changes of relevant protein markers. Described herein are biomarkers associated with neurodegenerative diseases and disorders as well as methods of detecting biomarkers associated with neurodegenerative diseases and disorders and using such biomarkers for making a clinical decision for an individual suspected of having or having the neurodegenerative disease or disorder.

### Certain Terminologies

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The terms “individual,” “patient,” or “subject” are used interchangeably. None of the terms require or are limited to a situation characterized by the supervision (e.g., constant or intermittent) of a health care worker (e.g., a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent, halt, or slow down (lessen) the progression of a targeted pathologic condition or disorder. Those in need of treatment include those already with the disorder, as well as those prone to have the disorder, or those in whom the disorder is to be prevented. For example, a subject or mammal is successfully “treated” for AD, if, after receiving a therapeutic amount of a therapeutic agent, the subject shows observable and/or measurable reduction or relief of, or absence of one or more symptoms of AD, reduced morbidity and/or mortality, and improvement in quality of life issues.

The term “antibody” as used herein refers to immunoglobulin molecules and immunologically active portions of immunoglobulin molecules, e.g., molecules that contain an antigen binding site that immunospecifically binds an antigen. The term also refers to antibodies comprised of two immunoglobulin heavy chains and two immunoglobulin light chains as well as a variety of forms including full length antibodies and portions thereof including, for example, an immunoglobulin molecule, a polyclonal antibody, a monoclonal antibody, a recombinant antibody, a chimeric antibody, a humanized antibody, a CDR-grafted antibody, F(ab)₂, Fv, scFv, IgGΔCH₂, F(ab′)₂, scFv2CH₃, F(ab), VL, VH, scFv4, scFv3, scFv2, dsFv, Fv, scFv-Fc, (scFv)2, a disulfide linked Fv, a single domain antibody (dAb), a “nanobody”, a diabody, a multispecific antibody, a dual specific antibody, an anti-idiotypic antibody, a bispecific antibody, any isotype (including, without limitation IgA, IgD, IgE, IgG, or IgM) a modified antibody, and a synthetic antibody (including, without limitation non-depleting IgG antibodies, T-bodies, or other Fc or Fab variants of antibodies).

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Methods of the Disclosure

Neurodegenerative diseases or disorders such as Parkinson's disease (PD) are complex diseases and disorders and determining a clinical decision in an individual having or suspected of having a neurodegenerative disease or disorder requires accurate and comprehensive biomarkers. Described herein are biomarkers associated with neurodegenerative diseases or disorders such as PD as well as methods of detecting biomarkers that are highly accurate and specific for use in making a clinical decision in an individual having or suspected of having a neurodegenerative disease or disorder.

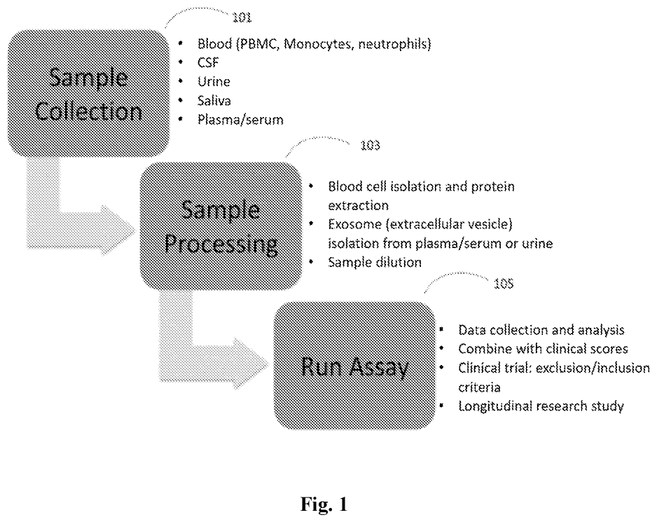

FIG. 1 depicts an exemplary schema of methods described herein. In a sample collection step 101, a biological sample is taken from an individual. The individual may have a neurodegenerative disease or disorder (e.g., Parkinson's disease). In some instances, the individual is suspected of having a neurodegenerative disease or disorder (e.g., Parkinson's disease). In some instances, the individual may have an underlying genetic predisposition to develop such a neurodegenerative disease or disorder. In some instances, the individual has a neurodegenerative disease or disorder selected from the group consisting of Parkinson's disease, multiple system atrophy (MSA); amyotrophic lateral sclerosis (ALS), progressive supranuclear palsy (PSP), corticobasal syndrome, Alzheimer's disease, frontotemporal dementia, multiple sclerosis (MS), corticobasal degeneration, motor neuron disease, spinocerebellar ataxia (SCA), spinal muscular atrophy (SMA), Huntington's disease (HD), Lewy body disease, Friedrich's ataxia, and synucleinopathies. The biological sample, in some instances, is selected from the group consisting of whole blood, specific blood cell subtypes, plasma, serum, urine, cerebrospinal fluid, tears, saliva, skin, and exosomes. In some instances, the biological sample is a sample that does not comprise exosomes. In some instances, the specific blood cell-subtypes is selected from the group consisting of peripheral blood mononuclear cells (PBMCs), neutrophils, lymphocytes, monocytes, eosinophils, basophils, and erythrocytes. The biological sample is then assayed in a sample processing step 103. In some instances, the sample processing step comprises blood cell isolation or exosome isolation from the biological sample. In some instances, exosomes are extracted from the group consisting of plasma, serum, urine, and cerebrospinal fluid. In some instances, the sample processing step comprises protein extraction. In some instances, the sample processing step comprises use of a surfactant (e.g., saponin) or detergent. In some instances, Following the sample processing step 103, the biological sample is then assayed for a biomarker in an assay step 105. In some instances, the biomarker is a level, post-translational modification, or both of LRRK2, alpha-synuclein, Rab8a, Rab10, Rab12, and Rab29. In some instances, the assay measures total protein, phosphorylated protein, or aggregated protein. In some instances, the aggregated protein is selected from the group consisting of amorphous aggregates, dimers, oligomers, fibrils, and combinations thereof. In some instances, the level is a concentration. In some instances, the level is a ratio of phosphorylation to total protein. In some instances, the LRRK2 is total LRRK2. In some instances, the LRRK2 is pS935-LRRK2. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some instances, total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are assayed. Various assays may be used. In some cases, the assay is selected from the group consisting of a Western blot, Dot blot, enzyme-linked immunosorbent assays (ELISA), single-molecule ELISA, chemiluminescence, absorbance, and chromatography. In some cases, the assay is an immunoassay. In some instances, the assay is an ELISA assay. In some cases, the assay detects at least or about 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6, 7, 8, 9, 10, 11, 12, 13, 14, 15, 16, 17, 18, 19, 20, or more than 20 microgram per milliliter (ug/mL) of protein. In some cases, the assay detects a range of about 0.0001 to about 10, 0.0001 to about 5, 0.0001 to about 1, 0.0001 to about 0.1, 0.0002 to about 10, 0.0002 to about 5, 0.0002 to about 1, 0.0002 to about 0.1, 0.0005 to about 10, 0.0005 to about 5, 0.0005 to about 1, 0.0005 to about 0.1, 0.001 to about 10, 0.001 to about 5, 0.001 to about 1, 0.001 to about 0.1, 0.005 to about 10, 0.005 to about 5, 0.005 to about 1, 0.0005 to about 0.1, 0.01 to about 10, 0.01 to about 5, 0.01 to about 1, 0.01 to about 0.1, 0.05 to about 10, 0.05 to about 5, 0.05 to about 1, or 0.05 to about 0.1. Data from the assay may be collected and analyzed. In some instances, the data from the assay is used with clinical scores. In some instances, a clinical decision is made based on the data, the clinical scores, or both. The level, post-translational modification, or both of the biomarker can be used for making a clinical decision. The results of the assay may be used to help determine a person's diagnosis, prognosis, medication treatment or prevention therapy, for the individual. In some instances, the clinical decision relates to drug development in a clinical trial. For example, the level, post-translational modification, or both of biomarker is used to inform a design of a clinical trial. Considerations for the design of the clinical trial may be selected from the group consisting of size, inclusion criteria, exclusion criteria, and combinations thereof. In some instances, the biomarker is used to predict a therapeutic response to a therapy. In some instances, the biomarker is used for informing a longitudinal research study. In some instances, the biomarker is used as an outcome of a clinical trial. In some instances, the biomarker is used as a surrogate endpoint in a clinical trial. The biomarker may be used for determining timing of administration of a treatment. The clinical trial may be selected from the group consisting of a preclinical trial, Phase I clinical trial, Phase II clinical trial, and Phase III clinical trial.

Disclosed herein are methods for making a clinical decision in an individual having, suspected of having, or at risk of progressing to a neurodegenerative disease or disorder, comprising: a) obtaining a biological sample from the individual having, suspected of having, or at risk of progressing to the neurodegenerative disease or disorder; b) performing an assay on the biological sample to determine a level, post-translational modification, or both of LRRK2, alpha-synuclein, Rab8a, Rab10, Rab12, and Rab29; and c) making the clinical decision based on the level, post-translational modification, or both of LRRK2, alpha-synuclein, Rab8a, Rab10, Rab12, and Rab29. In some instances, the LRRK2 is total LRRK2. In some instances, the LRRK2 is pS935-LRRK2. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some instances, total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are assayed.

Provided herein are methods for making a clinical decision for an individual having or suspected of having a neurodegenerative disease or disorder, wherein the clinical decision is determining a prognosis for the individual. Prognosis can comprise determining the outcome of a patient's disease, the chance of recovery, or how the disease will progress.

In some instances, the clinical decision comprises determining a likelihood or risk of the individual developing the neurodegenerative disease or disorder. In some instances, the likelihood or risk of developing the neurodegenerative disease or disorder is based on the level, post-translational modification, or both of a biomarker, wherein the likelihood of developing neurodegenerative disease or disorder is increased when the level, post-translational modification, or both of the biomarker is elevated compared to a reference level, post-translational modification, or both derived from a cohort of control individuals. In some instances, the biomarker is a level, post-translational modification, or both of one or more proteins selected from the group consisting of LRRK2, alpha-synuclein, Rab8a, Rab10, Rab12, and Rab29. In some instances, the biomarker is a level, post-translational modification, or both of LRRK2, alpha-synuclein, Rab8a, Rab10, Rab12, and Rab29. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some instances, the biomarker is one or more proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

In some instances, the likelihood or risk of developing the neurodegenerative disease or disorder is increased by at least or about 15%, 20%, 25%, 30%, 35%, 40%, 45%, 50%, 55%, 60%, 65%, 70%, 75%, 80%, 85%, 90%, 95% or more than 95% when the level, post-translational modification, or both of the biomarker is elevated compared to a reference level, post-translational modification, or both derived from a cohort of control individuals. In some instances, the likelihood or risk of developing the neurodegenerative disease or disorder is increased by at least or about 1.5×, 2×, 2.5×, 3×, 3.5×, 4.0×, 4.5×, 5×, 6×, 7×, 8×, 9×, 10×, or more than 10× when the level, post-translational modification, or both of the biomarker is elevated compared to a reference level, post-translational modification, or both derived from a cohort of control individuals. In some instances, the biomarker is a level, post-translational modification, or both of one or more proteins selected from the group consisting of LRRK2, alpha-synuclein, Rab8a, Rab10, Rab12, and Rab29. In some instances, the biomarker is a level, post-translational modification, or both of LRRK2, alpha-synuclein, Rab8a, Rab10, Rab12, and Rab29. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some instances, the biomarker is one or more proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

Methods as described herein for determining the likelihood or risk of developing the neurodegenerative disease or disorder, in some instances, are improved as compared to methods comprising neurological tests, mental exams, or brain imaging (e.g. MRI, CT, or PET scans).

In some instances, the clinical decision comprises determining a severity of the neurodegenerative disease or disorder. In some instances, the severity of the disease is correlated with the level, post-translational modification, or both of one or more proteins selected from the group consisting of LRRK2, alpha-synuclein, Rab8a, Rab10, Rab12, and Rab29. In some instances, the severity of the disease is correlated with the level, post-translational modification, or both of LRRK2, alpha-synuclein, Rab8a, Rab10, Rab12, and Rab29. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some instances, the severity of the disease is correlated with the level, post-translational modification, or both of one or more proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

Various neurodegenerative diseases or disorders are contemplated herein. In some embodiments, the neurodegenerative disease or disorder is selected from the group consisting of Parkinson's disease, multiple system atrophy (MSA); amyotrophic lateral sclerosis (ALS), progressive supranuclear palsy (PSP), corticobasal syndrome, Alzheimer's disease, frontotemporal dementia, multiple sclerosis (MS), corticobasal degeneration, motor neuron disease, spinocerebellar ataxia (SCA), spinal muscular atrophy (SMA), Huntington's disease (HD), Lewy body disease, Friedrich's ataxia, and synucleinopathies. In some embodiments, the neurodegenerative disease or disorder is Parkinson's disease. In some embodiments, Parkinson's disease is familial Parkinson's disease. In some embodiments, the Parkinson's disease is sporadic Parkinson's disease.

Described herein are methods for making a clinical decision in an individual, wherein the clinical decision is related to a clinical trial. In some instances, a biomarker is used for making the clinical decision relating to a clinical trial. In some instances, the biomarker is a level, post-translational modification, or both of one or more proteins selected from the group consisting of LRRK2, alpha-synuclein, Rab8a, Rab10, Rab12, and Rab29. In some instances, the biomarker is a level, post-translational modification, or both of LRRK2, alpha-synuclein, Rab8a, Rab10, Rab12, and Rab29. In some instances, the LRRK2 is pS935-LRRK2. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some instances, the biomarker is one or more proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

In some instances, the clinical decision comprises determining eligibility of the individual for a clinical trial. In some instances, the methods are used to design a clinical trial. In some instances, a level, post-translational modification, or both of the biomarker informs enrollment criteria of an individual in a clinical trial. The level, post-translational modification, or both of the biomarker may be used for determining size of the clinical trial, exclusion criteria, inclusion criteria, or combinations thereof. The therapy tested in the clinical trial can be a conventional treatment including a drug that has been licensed and approved by a regulatory agency to treat the disease or condition. Alternatively, the therapy tested in the clinical trial is a medicament that is a non-licensed or non-approved drug or drug candidate; or in some cases a licensed and approved medicament that is not being marketed in the same territory as the territory that the clinical trial is conducted.

Generally, clinical trials have been categorized into, among others, prevention trials (e.g., how to prevent initially or recurrence of a condition), screening trials (e.g., detection of a condition), diagnostic trials (e.g., study or compare tests or procedures for diagnosing a condition), treatment trials (e.g., test new treatments, therapeutics, combinations of such or new approaches of medical intervention), behavioral trials, quality of life trials (e.g., explore and measure or evaluate ways to improve comfort and/or quality of life), and compassionate use trials (e.g., expanded access or last resort, where no alternative effective treatments have been developed). In addition, trial designs might be categorized into, among others, fixed trials (e.g., where participants enter or leave trial, according to fixed criteria set by design), adaptive trials (e.g., where data generated during the trial are used to design the trial and interim data is used to modify trial as it proceeds; may modify, e.g., dosage, sample sizes, drug (therapeutic), patient selection criteria; often apply a Bayesian experimental design to assess the trial's progress), and “complex innovative design” (CID; including the use of adaptive, Bayesian, and other novel statistical approaches; see, e.g., US FDA CID pilot program and CID webpage, and counterpart descriptions used by other regulatory agencies as examples and strategies being used in these trials). Any or all of these features may be included herein.

Various features of clinical trials can include randomized (e.g., where participants are randomly assigned to various study arms), blinded (e.g., where participants do not know which of alternative treatments they receive), double blinded (e.g., where neither participants nor researchers know which of alternative treatments they receive), or double dummy (e.g., in alternating periods, with possible switching of (or between) treatments).

Methods as described herein for making a clinical decision in an individual based on a level, post-translational modification, or both of the biomarker may comprise determining a mechanism of action of a drug used in a clinical trial. In some instances, the level, post-translational modification, or both of the biomarker is used to understand why an individual or subset(s) of individuals respond well given their particular genetic makeup. In some instances, the level, post-translational modification, or both of the biomarker may be used to select an appropriate therapy for a given individual given her disease characteristics. In some instances, the level, post-translational modification, or both of the biomarker is used to select an appropriate therapy for a given individual when multiple therapies are available to choose from. In some instances, the level, post-translational modification, or both of the biomarker may be used to decide when not to select a particular therapy for a given individual given her disease characteristics. In some instances, the level, post-translational modification, or both of the biomarker is used to achieve clinical remission or excellent response when the patient is administered with a carefully chosen therapy, e.g., using a particular drug of choice at the first instance. In some instances, the clinical decision is measuring target engagement of an investigational compound. In some instances, the clinical decision is determining exclusion criteria, inclusion criteria, or both for a clinical trial of an investigational compound.

The level, post-translational modification, or both of the biomarker can be used for making a clinical decision, wherein the clinical decision comprises determining a drug dosing and schedule during a treatment cycle. In some instances, the level, post-translational modification, or both of the biomarker is used to determine therapeutic effectiveness. Therapeutic effectiveness may comprise how well the therapy, including the drug, worked in the individual or how well the individual responded to the treatment during and at the end of the treatment cycle. In some instances, the level, post-translational modification, or both of the biomarker is used to select an alternate therapeutic (drug) that can be considered as the next best choice based on, e.g., a mechanistic rationale, if the first choice failed to achieve reasonable therapeutic effectiveness.

In some instances, the level, post-translational modification, or both of the biomarker is used as a surrogate endpoint in a clinical trial. A surrogate endpoint of a clinical trial may be defined as laboratory measurement or a physical sign used as a substitute for a clinically meaningful endpoint that measures directly how a patient feels, functions, or survives. In some instances, the level, post-translational modification, or both of the biomarker is used as a surrogate endpoint for acceleration of approval of a treatment in the early clinical stages or preclinical stages of the disease.

The level, post-translational modification, or both of the biomarker can be used in a research study. In some embodiments, the research study is a longitudinal research study. In some embodiments, the research study is a cross-sectional research study.

The level, post-translational modification, or both of the biomarker may be used for determining whether a test predicts a therapeutic response to a treatment. For example, the level, post-translational modification, or both of the biomarker may be used to determine whether a positive or negative baseline test predicts a therapeutic response.

Described herein, in some instances, is selecting an individual for a clinical trial based on the level, post-translational modification, or both of the biomarker. In some instances, one or more biomarkers are used for selecting an individual for a clinical trial.

Following selection of individuals for a clinical trial based on the level, post-translational modification, or both of the biomarker, a change in the level, post-translational modification, or both of the biomarker can be monitored. In some instances, the change in the level, post-translational modification, or both of the biomarker is a reduction or an increase by at least 15%, 20%, 25%, 30%, 35%, 40%, 45%, 50%, 55%, 60%, 65%, 70%, 75%, 80%, 85%, 90%, 95% or more than 95%. In some instances, the change in the level, post-translational modification, or both of the biomarker is a reduction or an increase by at least or about 1.5×, 2×, 2.5×, 3×, 3.5×, 4.0×, 4.5×, 5×, 6×, 7×, 8×, 9×, 10×, or more than 10×. In some instances, the level, post-translational modification, or both of the biomarker is reduced or increased after 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years, or more than 2 years. In some instances, the change in the level, post-translational modification, or both of biomarker is reduced or increased by at least 15%, 20%, 25%, 30%, 35%, 40%, 45%, 50%, 55%, 60%, 65%, 70%, 75%, 80%, 85%, 90%, 95% or more than 95% after 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years, or more than 2 years. In some instances, the change in the level, post-translational modification, or both of the biomarker is reduced or increased by at least or about 1.5×, 2×, 2.5×, 3×, 3.5×, 4.0×, 4.5×, 5×, 6×, 7×, 8×, 9×, 10×, or more than 10× after 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years, or more than 2 years.

The change in the level, post-translational modification, or both of the biomarker can be monitored following a therapy for the neurodegenerative disease or disorder. In some instances, the therapy is selected from the group consisting of a small molecule, an immunotherapy, a gene therapy, a non-medication based therapy, an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic.

In some instances, the clinical decision comprises determining an endpoint for the therapy for the neurodegenerative disease or disorder. In some instances an individual's responsiveness to the therapy is based on the level, post-translational modification, or both of the biomarker. Determining the individual's responsiveness to the therapy may comprise determining a likelihood of an adverse response to the therapy. In some instances, determining the individual's responsiveness to the therapy comprises determining a likelihood of a beneficial response to the therapy.

Methods as described herein may be used for modifying the therapy based on the level, post-translational modification, or both of one or more proteins selected from the group consisting of LRRK2, alpha-synuclein, Rab8a, Rab10, Rab12, and Rab29. In some instances, the therapy is modified based on the level, post-translational modification, or both of LRRK2, alpha-synuclein, Rab8a, Rab10, Rab12, and Rab29. In some instances, the therapy is modified based on one or more proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. For example, the level, post-translational modification, or both of one or more proteins is measured a first time in the biological sample from the individual and then the level, post-translational modification, or both of one or more proteins is measured a second time after the individual has received the therapy. In some instances, measuring the level, post-translational modification, or both of one or more proteins is used to monitor the effectiveness of the therapy by monitoring for a change in the level, post-translational modification, or both of one or more proteins. The change can be an increase. In some instances, the change is a decrease.

Described herein, in some instances, are methods for determining when to introduce the therapy for the neurodegenerative disease or disorder based on the level, post-translational modification, or both of the biomarker. The level, post-translational modification, or both of the biomarker may be used to determine a decision selected from the group consisting of introduce a treatment to improve symptoms, slow the clinical progression, prevent the clinical onset of the neurodegenerative disease or disorder, and combinations thereof.

In some instances, the individual selected for a clinical trial based on the level, post-translational modification, or both of a biomarker exhibits symptoms of the neurodegenerative disease or disorder. In some instances, the symptoms comprise cognitive change. Exemplary cognitive symptoms include, but are not limited to, mental decline, difficulty thinking and understanding, confusion in the evening hours, delusion, disorientation, forgetfulness, making things up, mental confusion, difficulty concentrating, inability to create new memories, inability to do simple math, and inability to recognize common things. In some instances, the symptoms comprise dementia. In some instances, the symptoms are behavioral. In some instances, the behavioral symptoms are selected from the group consisting of aggression, agitation, difficulty with self-care, irritability, meaningless repetition of own words, personality changes, restlessness, lack of restraint, and wandering and getting lost. The symptoms can comprise changes in mood such as anger, apathy, general discontent, loneliness, and mood swings. In some instances, the individual selected for a clinical trial based on the level, post-translational modification, or both of the biomarker is unimpaired.

In some instances, the therapy is administered based on the level, post-translational modification, or both of the biomarker. In some instances, the biomarker is one or more proteins selected from the group consisting of LRRK2, alpha-synuclein, Rab8a, Rab10, Rab12, and Rab29. In some instances, the therapy is administered based on the level, post-translational modification, or both of LRRK2, alpha-synuclein, Rab8a, Rab10, Rab12, and Rab29. In some instances, the therapy is optimized based on the level, post-translational modification, or both of LRRK2, alpha-synuclein, Rab8a, Rab10, Rab12, and Rab29. In some instances, the level, post-translational modification, or both of LRRK2, alpha-synuclein, Rab8a, Rab10, Rab12, and Rab29 is measured prior to a treatment, during a treatment, or after a treatment. For example, the level, post-translational modification, or both of LRRK2, alpha-synuclein, Rab8a, Rab10, Rab12, and Rab29 is measured at 1 day, 2 days, 3 days, 4 days, 5 days 6 days, 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years, or more than 2 years before treatment. In some instances, the level, post-translational modification, or both of LRRK2, alpha-synuclein, Rab8a, Rab10, Rab12, and Rab29 is measured at 1 day, 2 days, 3 days, 4 days, 5 days 6 days, 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years, or more than 2 years occurs after treatment.

Methods as described herein can comprise performing an assay on a biological sample. In some instances, the biological sample is selected from the group consisting of whole blood, specific blood cell subtypes, plasma, serum, urine, cerebrospinal fluid, tears, saliva, skin, and exosomes. In some instances, the biological sample is a sample that does not comprise exosomes. In some instances, the biological sample is a cerebrospinal fluid sample that does not comprise exosomes. In some instances, the specific blood cell subtypes is selected from the group consisting of peripheral blood mononuclear cells (PBMCs), neutrophils, lymphocytes, monocytes, eosinophils, basophils, and erythrocytes. The biological sample can be a blood sample obtained by a venous blood draw. The biological sample can be a blood sample obtained from a finger prick blood draw. The biological can be obtained by a health care provider or by the subject. The method can comprise obtaining the biological sample from a subject. In some cases, the biological sample is obtained from the subject during a visit to the clinic or the hospital.

The biological sample may be processed in a sample processing step prior to analysis. In some embodiments, the biological sample is diluted prior to analysis. In some embodiments, cells are extracted from the biological sample. In some embodiments, exosomes are extracted from the biological sample. In some instances, exosomes are extracted from the group consisting of plasma, serum, urine, and cerebrospinal fluid. In some embodiments, protein is extracted from the biological sample. In some embodiments, nucleic acid is extracted from the biological sample. In some embodiments, the nucleic acid is DNA. In some embodiments, the nucleic acid is RNA.

In some embodiments, the biological sample is processed using an organic solvent, a surfactant, or hypotonic water. In some embodiments, the biological sample is processed using an organic solvent, a surfactant, or hypotonic water to release the protein from exosomes. In some embodiments, said organic solvent comprises one or more of: methanol, acetone, phenol, benzene, and toluene. In some embodiments, said surfactant comprises one or more of: saponin, Triton X-100, Tween-20, 3-([3-cholamidopropyl] dimethylammonio)-1-propanesulfonate hydrate (CHAPS) cell extract buffer, NP-40, SDS, Span 80, and Digitonin. In some embodiments, said surfactant comprises saponin. In some embodiments, said hypotonic water is ultrapure nuclease-free water.

The protein or nucleic acid, in some embodiments, is extracted using any technique that does not interfere with subsequent analysis. For example, the nucleic acid is extracted using alcohol precipitation, using ethanol, methanol, or isopropyl alcohol, phenol, chloroform, cesium chloride, or in combination thereof. In some embodiments, the nucleic acid is extracted using sodium, potassium or ammonium acetate or any other salt commonly used to precipitate DNA. In some embodiments, the nucleic acid is extracted using a column or resin based nucleic acid purification. In some embodiments, the protein is extracted following lysis of the cells. In some cases, cells are lysed using a buffer such as RIPA (RadiolmmunoPrecipitation Assay) buffer, an NP-40 lysis buffer, a sodium dodecyl sulfate (SDS) lysis buffer, an ammonium-chloride-potassium (ACK) lysis buffer, or other lysis buffer. Denaturing buffers, such as a buffer which can comprise urea, dithiothreitol, or other denaturing agent, may also be used. The sample may be filtered or centrifuged to remove lipids and particulate matter. The sample may also be purified to remove nucleic acids, or may be treated with RNases and DNases. In some embodiments, after extraction the nucleic acid or protein is stored. In some instances, the nucleic acid is stored in water, Tris buffer, or Tris-EDTA buffer before subsequent analysis. For example, storage is less than 8° C., 4° C., −20° C., or −70° C. In some embodiments, the nucleic acid or protein is stored for 1, 2, 3, 4, 5, 6, or 7 days. In some embodiments, the nucleic acid or protein is stored for 1, 2, 3, or 4 weeks. In some embodiments, the nucleic acid or protein is stored for 1, 2, 3, 4, 5, 6, 7, 8, 9, 10, 11, or 12 months. In some embodiments, the nucleic acid or protein is diluted prior to analysis.

In another aspect, the method includes performing a plurality of assays on the biological sample from the individual. The plurality of assays performed on the biological sample may comprise detecting the level, post-translational modification or both of a plurality of biomarkers. In some cases, the plurality of biomarkers comprises two or more, three or more, or four or more biomarkers. In some cases, the plurality of biomarkers comprises three, four, five, six, seven, eight, nine, ten, eleven, twelve, thirteen, fourteen, fifteen, sixteen, or more than sixteen biomarkers. In some cases, the plurality of biomarkers comprises two biomarkers. In some cases, the plurality of biomarkers comprises three biomarkers. In some cases, the plurality of biomarkers comprises four biomarkers. In some cases, the plurality of biomarkers comprises five biomarkers. In some cases, the plurality of biomarkers comprises six biomarkers. In some cases, the plurality of biomarkers comprises seven biomarkers. In some cases, the plurality of biomarkers comprises eight biomarkers. In some cases, the plurality of biomarkers comprises nine biomarkers. In some cases, the plurality of biomarkers comprises ten biomarkers. In some cases, the plurality of biomarkers comprises eleven biomarkers. In some cases, the plurality of biomarkers comprises twelve biomarkers. In some cases, the plurality of biomarkers comprises thirteen biomarkers. In some cases, the plurality of biomarkers comprises fourteen biomarkers. In some cases, the plurality of biomarkers comprises fifteen biomarkers. In some cases, the plurality of biomarkers comprises sixteen biomarkers. In some cases, the plurality of biomarkers comprises seventeen biomarkers. In some cases, the plurality of biomarkers comprises eighteen biomarkers. In some cases, the plurality of biomarkers comprises nineteen biomarkers. In some cases, the plurality of biomarkers comprises twenty biomarkers.

In some aspects, the method involves performing a plurality of assays on a biological sample obtained from the subject and detecting the level, post-translational modification, or both of the plurality of biomarkers present in the biological sample. The plurality of assays can be performed in different reactions. In one example, the different reactions can be carried out in different wells of a microplate. The plurality of assays can be performed in the same reaction. In some instances, the plurality of assays are performed in a single well, spot, or microplate. In one example, the same reaction can comprise multiple different capture antibodies. Alternatively, the plurality of assays can comprise at least one reaction detecting a single biomarker and at least one reaction detecting one or more biomarkers. The plurality of assays can be a plurality of immunoassays.

Described herein, in some embodiments, are assays for detecting a level, post-translational modification, or both of a protein. In some instances, the assay measures total protein. In some instances, the assay measures a post-translational modification. Exemplary post-translational modifications include, but are not limited to, glycosylation, carboxylation, deamidation, oxidation, hydroxylation, 0-sulfation, amidation, glycylation, glycation, alkylation, acylation, acetylation, phosphorylation, biotinylation, formylation, lipidation, iodination, prenylation, oxidation, palmitoylation, phosphatidylinositolation, phosphopantetheinylation, sialylation, and selenoylation. In some instances, the post-translational modification is phosphorylation. In some instances, the post-translational modification is autophosphorylation at specific sites. In some instances, the assay measures aggregated protein. Aggregated protein, in some instances, comprises pathological forms of the protein. In some instances, the aggregated protein is selected from the group consisting of amorphous aggregates, oligomers, fibrils, and combinations thereof. In some instances, the aggregated protein comprises dimers of protein. In some instances, the dimers are propagating. In some instances, the dimers are non-propagating. In some instances, aggregate protein comprises oligomers of protein. Oligomers, in some instances, comprise two or more molecules of the protein interacting together. In some instances, the oligomers are chain-like. In some instances, the oligomers are spherical. In some instances, the level is a concentration. In some instances, the level is an amount of protein. In some instances, the level is a ratio of a post-translationally modified protein to total protein. For example, the level is a ratio of phosphorylation to total protein.

In some instances, the biomarker is a level, post-translational modification, or both of one or more proteins selected from the group consisting of LRRK2, alpha-synuclein, Rab8a, Rab10, Rab12, and Rab29. In some instances, the biomarker is a level, post-translational modification, or both of LRRK2, alpha-synuclein, Rab8a, Rab10, Rab12, and Rab29. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some instances, the biomarker is one or more proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

Aggregated alpha-synuclein, in some instances, comprises pathological forms of the protein. In some instances, aggregated-alpha synuclein comprises dimers of alpha-synuclein. In some instances, the dimers of alpha-synuclein are propagating. In some instances, the dimers of alpha-synuclein are non-propagating. In some instances, aggregated-alpha synuclein comprises oligomers of alpha-synuclein. Oligomers, in some instances, comprise two or more molecules of the protein interacting together. In some instances, the oligomers are chain-like. In some instances, the oligomers are spherical. In some instances, the aggregated alpha-synuclein comprise small (non-amyloid) fibrils. In some instances, the aggregated alpha-synuclein comprises amyloid-like fibrils. The mature fibrils can be characterized by specific n-sheet conformations as revealed by X-ray diffraction and circular dichroism patterns.

Methods as described herein, in some embodiments, comprise determining a level of at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least three proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least four proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least five proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least six proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least seven proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least eight proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least nine proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least ten proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least eleven proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least twelve proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of at least thirteen proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods as described herein comprise determining a level of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

A plurality of assays can comprise performing a test on the individual. In some instances, the test is a physical test. In some instances, the test is neurological test. In some instances, the assay comprises performing a cognitive test. The assay may comprise a test selected from the group consisting of a physical examination, a laboratory test, a neurological test of balance, a neurological test of vision, a neuropsychological test of memory, a neuropsychological test of problem solving, a neuropsychological test of language skills, and combinations thereof. In some instances, the methods described herein further comprise determining the individual's medical history. In some instances, the test is selected from the group consisting of the Unified Parkinson's Disease Rating Scale (UPDRS), the Hoehn and Yahr scale, Tinetti Test, the Barrow Neurological Institute (BNI) balance scale, the Romberg Test, the Turning test, the Standing On One Leg test, and the Tandem gait test.

In some cases, the method comprises detecting a level, post-translational modification, or both of the plurality of biomarkers in the individual at a plurality of time points. In some instances, the plurality of time points comprises two, three, four, five, six, seven, eight, nine, ten, or more than ten time points. In some instances, at least one time point of a plurality of time points comprises a time point before said individual has been treated with the therapy for the neurodegenerative disease or disorder. In some instances, at least one time point of a plurality of time points comprises a time point after said individual has been treated with the therapy for the neurodegenerative disease or disorder.

In some cases, the assay comprises an immunoassay or a ligand assay. In some cases, the assay is selected from the group consisting of enzyme-linked immunosorbent assay (ELISA), a colorimetric immunoassay, a homogeneous immunoassay, a non-optical immunoassay, a fluorescence immunoassay, a chemiluminescence immunoassay, an electro-chemiluminescence immunoassay, a fluorescence resonance energy transfer (FRET) immunoassay, a time resolved fluorescence immunoassay, a lateral flow immunoassay, a microspot immunoassay, a surface plasmon resonance assay, a ligand assay, a clotting assay, a chromatography assay, and immunocapture coupled with mass spectrometry. In some cases, the assay comprises an immunoassay. In some cases, the assay is selected from the group consisting of a Western blot, Dot blot, enzyme-linked immunosorbent assays (ELISA), chemiluminescence, absorbance, and chromatography. In some cases, the immunoassays are single-plexed. In some cases, the immunoassays are dual-plexed. In some cases, the immunoassays are multiplexed.

Methods as described herein can comprise a plurality of immunoassays. In some cases, the plurality of immunoassays are the same immunoassay (e.g., four or more ELISA assays). When the plurality of immunoassays are the same immunoassay, each of the plurality of immunoassays can detect a different biomarker. When the plurality of immunoassays are the same immunoassay, each of the plurality of immunoassays can be performed in the same reaction chamber or a different reaction chamber. In some instances, the plurality of immunoassays are performed in the same reaction chamber. A reaction chamber can be any suitable space for performing an immunoassay. Examples of reaction chambers include, but are not limited to, a well in a microplate, a microspin tube, a conical tube, or a droplet.

In some cases, the plurality of immunoassays are different immunoassays. When the plurality of immunoassays are different immunoassays, each of the plurality of immunoassays can detect a different biomarker. When the plurality of immunoassays are different immunoassays, each of the plurality of immunoassays can be performed in the same reaction chamber or a different reaction chamber. In some instances, the plurality of immunoassays are performed in the same reaction chamber.

Methods as described herein, in some embodiments, comprise a plurality of immunoassays for simultaneously detecting the level, post-translational modification, or both of one or more proteins selected from the group consisting of LRRK2, alpha-synuclein, Rab8a, Rab10, Rab12, and Rab29. In some embodiments, methods described herein comprise a plurality of immunoassays for simultaneously detecting the level, post-translational modification, or both of LRRK2, alpha-synuclein, Rab8a, Rab10, Rab12, and Rab29. In some instances, the LRRK2 is pS1292-LRRK2. In some instances, the alpha-synuclein is total alpha-synuclein. In some instances, the alpha-synuclein is pS129 alpha-synuclein. In some instances, the alpha-synuclein is aggregated alpha-synuclein. In some instances, the Rab8a is total Rab8a. In some instances, the Rab8a is pT72-Rab8a. In some instances, the Rab10 is total Rab10. In some instances, the Rab10 is pT73-Rab10. In some instances, the Rab12 is total Rab12. In some instances, the Rab12 is pS106-Rab12. In some instances, the Rab29 is total Rab29. In some instances, the Rab29 is pT71-Rab29. In some embodiments, methods described herein comprise a plurality of immunoassays for simultaneously detecting one or more proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, methods described herein comprise a plurality of immunoassays for simultaneously detecting total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

The plurality of assays, in some embodiments, are performed within a single reaction chamber. In some instances, the reaction chamber is a well in a microplate, a microspin tube, a conical tube, or a droplet. In some instances, at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least three proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least four proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least five proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least six proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least seven proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least eight proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least nine proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least ten proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least eleven proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least twelve proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, at least thirteen proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. In some embodiments, total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are detected within the single reaction chamber. Detection of the proteins can occur simultaneously.

Described herein, in some embodiments, are methods for detecting one or more proteins selected from the group consisting total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 using antibodies. In some embodiments, the antibodies directed to the one or more proteins selected from the group consisting total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29 are micro-spotted on an array. In some instances, the array is within a well. For example, the array is within a well of a microwell assay plate. The microwell assay plate may be a 6-well, 12-well, 24-well, 48-well, 96-well, or 384-well microwell assay plate.

The antibodies can be used in an immunoassay such as an ELISA. In some instances, the antibodies are capable of capturing one or more proteins selected from the group consisting total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some instances, secondary antibodies are used to detect the antibodies that detect the one or more proteins selected from the group consisting total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

In some instances, the antibodies or secondary antibodies are biotinylated, fluorescent, or enzyme conjugated. In some embodiments, the antibodies or secondary antibodies are labeled with a label selected from the group consisting of horseradish peroxidase (HRP), alkaline phosphatase (AP) or glucose oxidase, Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, BODIPY® FL, Coumarin, Cy®3, Cy®5, Fluorescein (FITC), Oregon Green®, Pacific Blue™, Pacific Green™, Pacific Orange™, Tetramethylrhodamine (TRITC), Texas Red®, 32P, 33P, 3H, 14C, and 1251.

Various types of antibodies can be used for the immunoassay. In some embodiments, the antibody comprises an IgG constant domain. In some embodiments, the antibody comprises an IgG1, IgG2, IgG3, or IgG4 constant domain, or a variant thereof. In some embodiments, the antibody is a monoclonal antibody. In some embodiments, the antibody is an antigen binding fragment. In some embodiments, the antibody is a Fab fragment, F(ab′)2 fragment, single chain Fv (scFv), diabody, triabody, or minibody. In some embodiments, the antibody is human. In some embodiments, the antibody is humanized.

The ELISA assay described herein, in some embodiments, detects at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least three proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least four proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least five proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least six proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least seven proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least eight proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least nine proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least ten proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least eleven proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least twelve proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, the ELISA assay detects at least thirteen proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29. In some embodiments, total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

In some cases, the assay comprises a non-immunoassay. In some cases, the assay is selected from the group consisting of High Performance Liquid Chromatography (HPLC), High Performance Liquid Chromatography Mass spectrometry (HPLC-MS), Gas Chromatography Mass Spectrometry (GC-MS), Liquid Chromatography Mass spectrometry (LC-MS), Liquid Chromatography Tandem Mass spectrometry (LC-MS/MS), immunohistochemistry (IHC), polymerase chain reaction (PCR), quantitative PCR (qPCR), and combinations thereof.

The assays (e.g., ELISA) described herein may detect various levels of proteins. In some cases, the assay detects at least or about 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6, 7, 8, 9, 10, 11, 12, 13, 14, 15, 16, 17, 18, 19, 20, or more than 20 microgram per milliliter (ug/mL) of protein. In some cases, the assay detects a range of about 0.0001 to about 10, 0.0001 to about 5, 0.0001 to about 1, 0.0001 to about 0.1, 0.0002 to about 10, 0.0002 to about 5, 0.0002 to about 1, 0.0002 to about 0.1, 0.0005 to about 10, 0.0005 to about 5, 0.0005 to about 1, 0.0005 to about 0.1, 0.001 to about 10, 0.001 to about 5, 0.001 to about 1, 0.001 to about 0.1, 0.005 to about 10, 0.005 to about 5, 0.005 to about 1, 0.0005 to about 0.1, 0.01 to about 10, 0.01 to about 5, 0.01 to about 1, 0.01 to about 0.1, 0.05 to about 10, 0.05 to about 5, 0.05 to about 1, or 0.05 to about 0.1.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Levels, Phosphorylation, and Activity of Parkinson's Disease Proteins LRRK2, Rab GTPases, and Alpha-Synuclein

An ELISA-based assay was performed to quantify changes in select proteins involved in neurodegenerative diseases such as Parkinson's disease.

The proteins in Table 1 are chosen to create a signature of changes in neurodegenerative diseases such as Parkinson's disease.

TABLE 1

| Protein | | |
|---|---|---|
| target | Analyte name | Specific modification |
| LRRK2 | Total LRRK2 | Total levels of the protein LRRK2 |
| | pSer935-LRRK2 | Phosphorylated LRRK2 at Ser935 |
| | pS1292-LRRK2 | Phosphorylated LRRK2 at Ser1292 |
| alpha- | Total alpha-synuclein | Total levels of the protein alpha- |
| synuclein | | synuclein |
| | pS129 alpha-synuclein | Phosphorylated LRRK2 at Ser129 |
| | Oligomeric/aggregated | Total levels of oligomeric or |
| | alpha-synuclein | aggregated alpha-synuclein |
| Rab | Total Rab8a | Total levels of the protein Rab8a |
| GTPase | pT72-Rab8a | Phosphorylated Rab8a at Thr72 |
| | Total Rab10 | Total levels of the protein Rab10 |
| | pT73-Rab10 | Phosphorylated Rab10 at Thr73 |
| | Total Rab12 | Total levels of the protein Rab12 |
| | pS106-Rab12 | Phosphorylated Rab12 at Ser106 |
| | Total Rab29 | Total levels of the protein Rab29 |
| | pT71-Rab29 | Phosphorylated Rab29 at Thr71 |

As seen in Table 2, changes in LRRK2 and Rab10 were observed in multiple single-plex biomarker assays employed in clinical studies of two independent PD cohorts.

TABLE 2
Summary of Biomarker Data

| | | In Vitro | | pS1292- | pT73 |
|---|---|---|---|---|---|
| | Total | Kinase | pS935- | LRRK2 | Rab10 |
| | LRRK2 | Activity | LRRK2 | (WB) | (WB) |
| Healthy | Baseline | Baseline | Baseline | Detectable; | Baseline |
| Control | | | | not | |
| (HC) | | | | quantitative | |
| Idiopathic | Like HC | Like HC | Elevated | Detectable; | Elevated |
| PD | | | *2 cohorts | not | |
| | | | | quantitative | |
| G2019S + | Like HC | Elevated | Slight | Detectable; | Like HC |
| PD− | | | decrease | not | |
| | | | | quantitative | |
| G2019S + | Like HC | Elevated | Slight | Detectable; | Elevated |
| PD+ | | | decrease | not | |
| | | | | quantitative | |
| A53T + | Like HC | Like HC | Like HC | Detectable; | Like HC |
| PD+ | | | | not | |
| | | | | quantitative | |

*HC: Healthy control; WB: Western immunoblot; PD: Parkinson's disease.

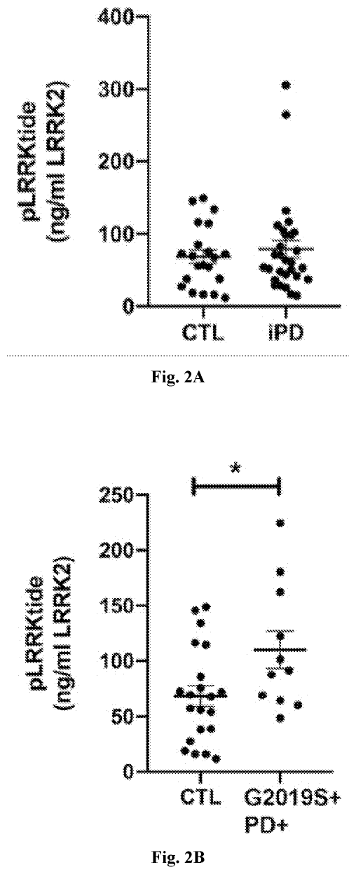

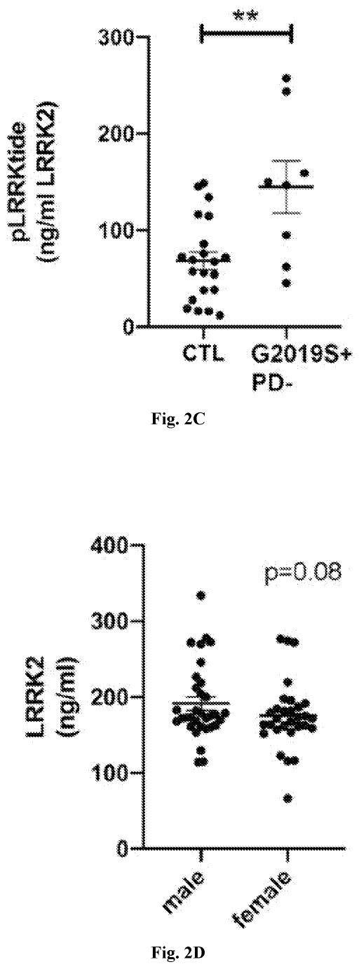

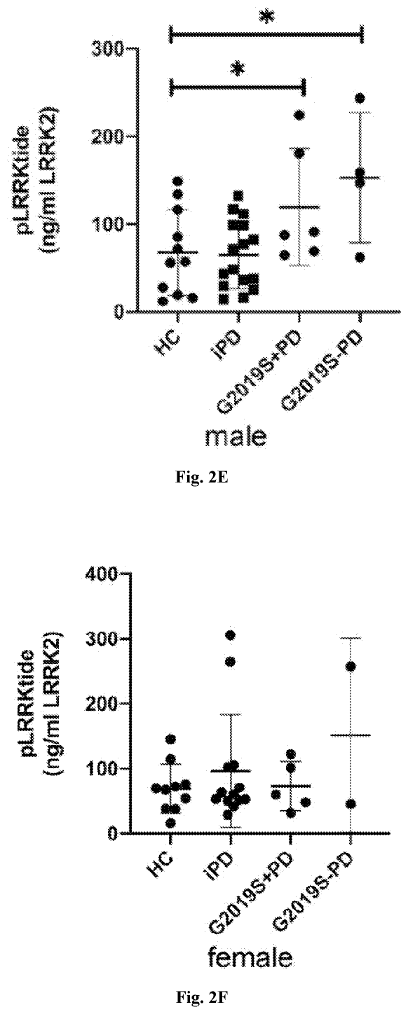

In vitro kinase activity using a model peptide substrate in an in-well kinase reaction was measured. The phosphorylation of the peptide was normalized to the total amount of LRRK2 in each well. No significant change in the intrinsic kinase activity of LRRK2 in PBMCs of iPD patients (FIG. 2A) was observed. The phosphorylation was significantly increased in affected (FIG. 2B) and healthy (FIG. 2C) carriers of the G2019S-LRRK2 mutation. Mann-Whitney U; * p<0.05, ** p<0.01. LRRK2 expression and in vitro activity were compared in PBMCs from males and females (FIG. 2D). In samples from healthy control and iPD patients, a trend for higher levels of expression in PBMCs from males compared to females was observed; Mann-Whitney U, p=0.07 (Ctl) and p=0.09 (iPD). Interestingly, the increased in vitro kinase activity detected in PBMCs from carriers of the G2019S-LRRK2 mutation, when analyzed separately by sex, was only evident in PBMCs from male subjects (FIG. 2E). The activity of LRRK2 from PBMCs of female G2019S carriers was not different from healthy controls (FIG. 2F). * p<0.05. The data shows LRRK2 in vitro kinase activity in PBMC extracts is increased in carriers of the G2019S mutation.

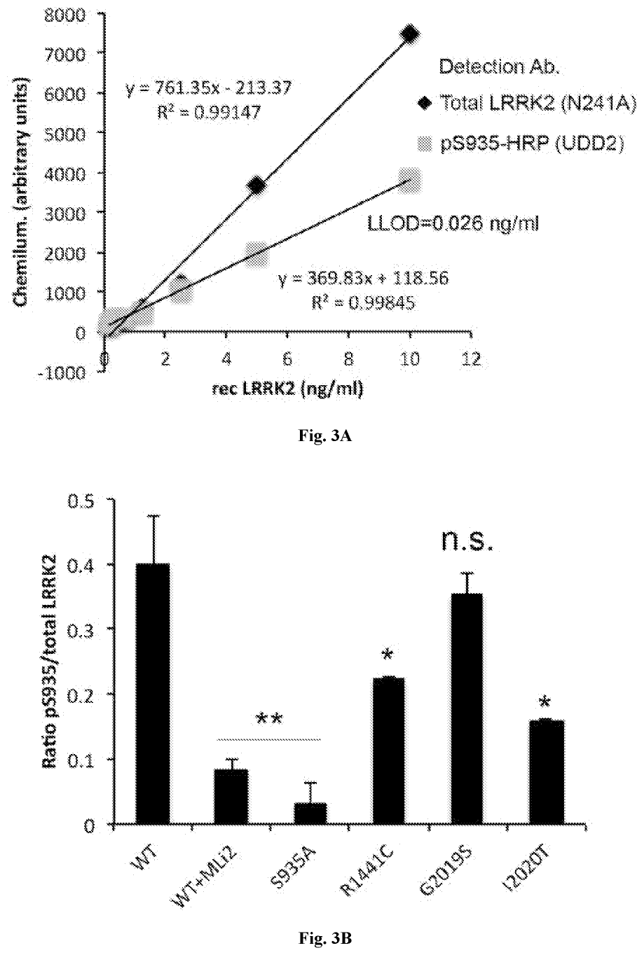

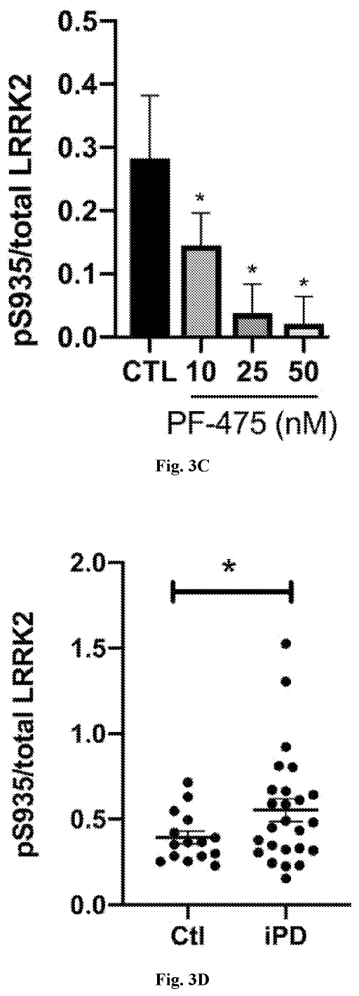

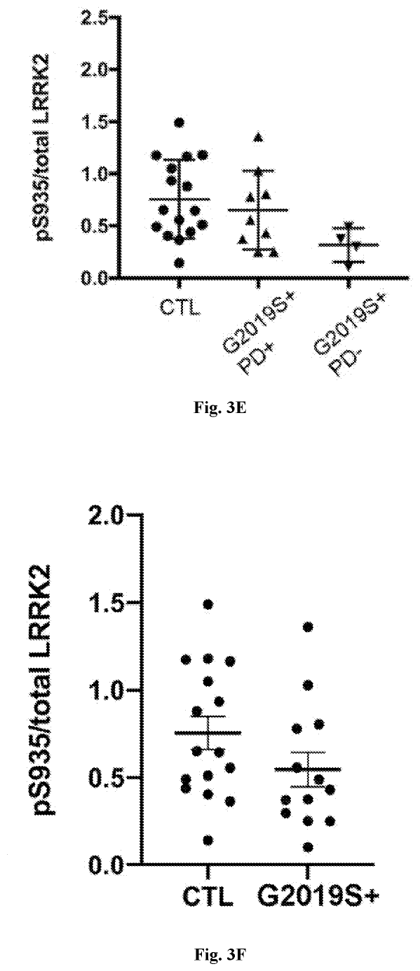

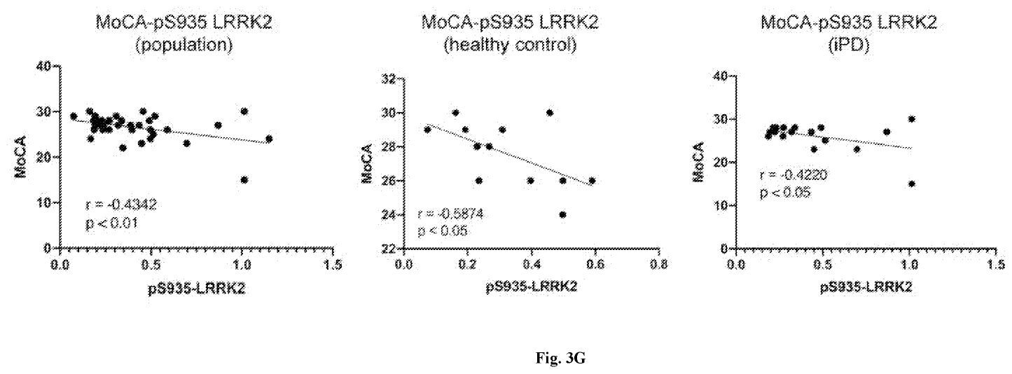

Phosphorylation of LRRK2 at the Ser935 residue by ELISA was assessed. For each sample, the signal corresponding to pS935-LRRK2 was normalized to its corresponding value of total LRRK2 (in ng/ml). Increasing amounts of recombinant human full-length LRRK2 was bound to parallel ELISA plates using the c41-2 clone for capture, and detected using total (N241A; for normalization) or phosphorylated (pS935-LRRK2, UDD2) antibodies (FIG. 3A). Extracts of HEK293T cells over-expressing WT (+/−treatment with MLi-2; 100 nM) or S935A-LRRK2 were subjected to ELISA using the anti-pS935-LRRK2 antibody either as a capture or detector antibody (FIG. 3B). In both cases, the signal was significantly lost for S935A-LRRK2 or following de-phosphorylation with the MLi-2 kinase inhibitor. Freshly isolated PBMCs from healthy volunteers were treated with increasing concentrations of the kinase inhibitor PF-475 for 1 hour, and subjected to pS935-LRRK2 ELISA (FIG. 3C). A significant dose-dependent loss of phosphorylated LRRK2 was observed. ANOVA, Tukey post-hoc comparisons; * p<0.05, ** p<0.01. pS935-LRRK2 levels in PBMCs of patients with iPD were significantly higher compared to healthy controls (FIG. 3D); * Mann-Whitney U, p<0.05. In contrast, in PBMC extracts from carriers of the G2019S mutation, with and without PD, there was a non-significant trend towards lower levels of pS935-LRRK2 compared to healthy controls (FIG. 3E). The values of all G2019S carriers are pooled for comparison against healthy controls (FIG. 3F). Levels of pS935-LRRK2 and various clinical parameters were compared (FIG. 3G). In iPD patients, there was no correlation between pS935-LRRK2 and the motor function scale UPDRS-III (not shown); however, performance on the cognitive function test, MoCA, was negatively correlated with pS935-LRRK2 levels in iPD patients (FIG. 3G; right panel) and healthy controls (FIG. 3G; center panel).

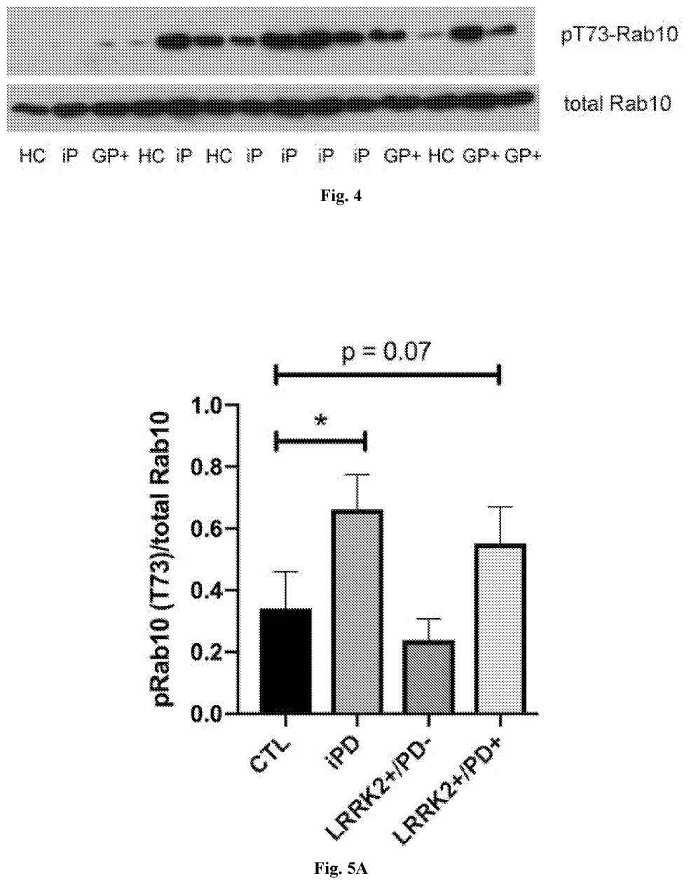

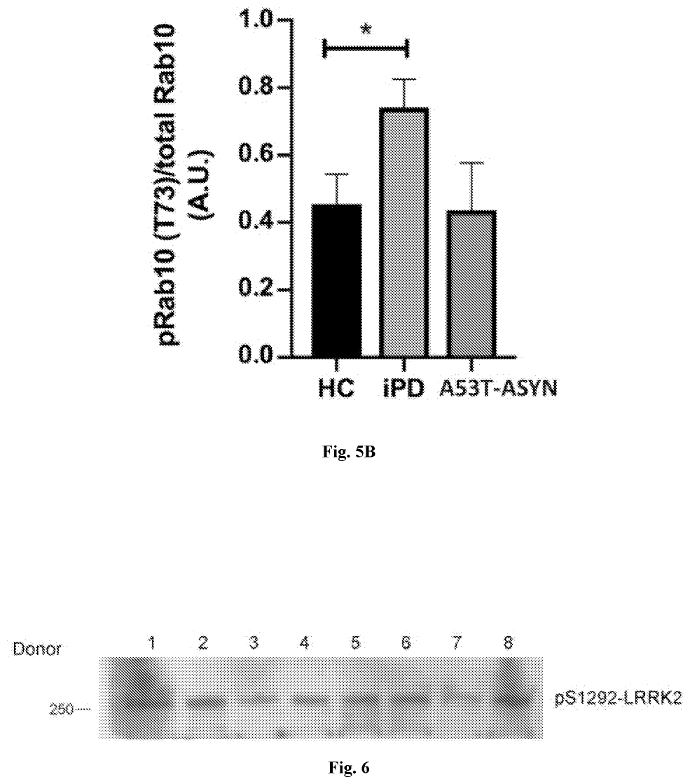

Phosphorylation of Rab10 at Thr73 in extracts of PBMCs obtained from healthy control subjects, or subjects with idiopathic PD; or carriers of the G2019S LRRK2 mutation, with and without PD were measured. Data from Western immunoblot analysis is seen in FIG. 4. The lower blot shows the same samples probed for total levels of Rab10. Band intensities of phosphorylated Rab10 were normalized to total levels of the protein. FIGS. 5A-5B show quantification of band intensity of Western immunoblots from PBMC extracts. Phosphorylated Rab10-Thr73 band intensities were normalized to bands reflecting total Rab10 protein levels. The data shows that phosphorylation of the LRRK2 substrate Rab10 (at Thr73) was significantly increased in the idiopathic PD group, as well as the carriers of the LRRK2 mutation with PD.

Western immunoblot assays were performed on PBMC extracts selected from each group, to show expression of pS1292-LRRK2. In order to detect this target in PBMC extracts, a signal boost reagent is added to the antibody solution; highlighting the need for more sensitive and quantitative measures. Data is seen in FIG. 6.

These data show that changes in LRRK2, alpha-synuclein, and Rab GTPases are linked to neurodegenerative disease and progression.

### Example 2: Cerebrospinal Fluid (CSF) Testing Using Multiplexed LRRK2 ELISA

This example assays whether LRRK2 present in CSF is detectable by ELISA and whether a dual-plex ELISA, comprised of 2 of the 14 planned antibody targets, could detect total and phosphorylated LRRK2.

Briefly, an ELISA assay was designed to contain antibody spots that capture total LRRK2 (clone c41-2; Melachroinou et al., 2020; or clone MC.028.83.76.242) and pS935-LRRK2 (clone UDD2; see again Melachroinou et al., 2020). As a detection reagent, in this dual-plex form capturing only LRRK2, the antibody was against total LRRK2 (clone UDD3), conjugated to biotin. Levels of pS935-LRRK2 and total LRRK2 were measured. The multiplexed format allows for the accurate normalization of phospho/total LRRK2 as both are quantified within the same well. Saponin treatment was to used to release exosomal LRRK2.

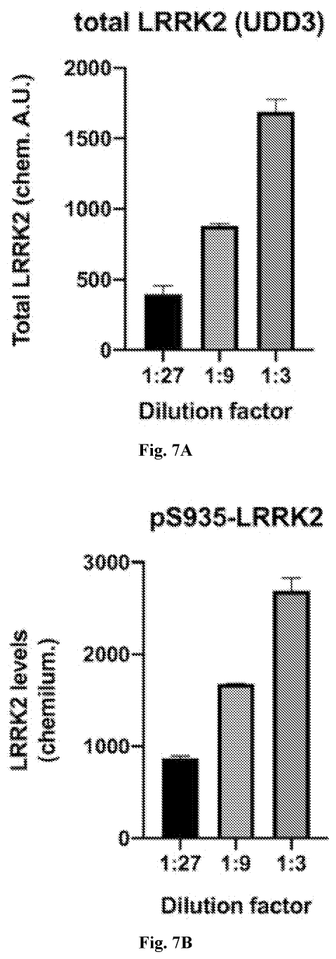

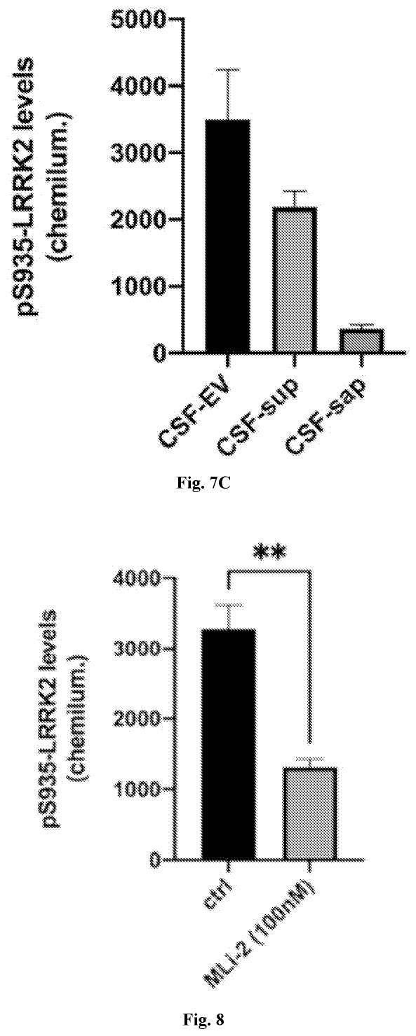

FIG. 7A demonstrates total LRRK2. FIG. 7B demonstrates pS935-LRRK2. As seen in FIG. 7C, the final levels of pS935-LRRK2 from the different CSF sample conditions are observed including for cerebrospinal fluid extracellular vesicles (CSF-EV), cerebrospinal fluid supernatant with exosomes depleted (CSF-sup), and cerebrospinal fluid saponin treated (CSF-sap). The largest signal derived from the extract of isolated (ultracentrifugation) exosomes; however significant signal was also seen in the supernatant from this ultracentrifugation. This is a strong indication that there is detectable phosphorylated LRRK2 “free” (not in the exosomes) in the CSF.

Experiments were performed using fresh PBMCs from healthy volunteer donors incubated with MLi2 (100 nM; 3 hr), which is a potent selective inhibitor of LRRK2 kinase activity. PBMC extracts were then diluted and incubated in the dual-plex test plate (measuring total and pS935-LRRK2). The signal obtained from the pS935-LRRK2 antibodies was then normalized to the corresponding signal from total LRRK2. Inhibition of LRRK2 activity with MLi2 induces a dramatic loss of phosphorylation at the Ser935 residue in LRRK2 (FIG. 8). As a positive control for the assay, cryopreserved PBMCs were incubated with vehicle or the kinase inhibitor MLi-2 (100 nM; 3 hr) and then processed the cells for the dual-plex ELISA prototype. A two-thirds reduction in pS935-LRRK2 levels in cells treated with MLi-2 compared to control cells (FIG. 8).

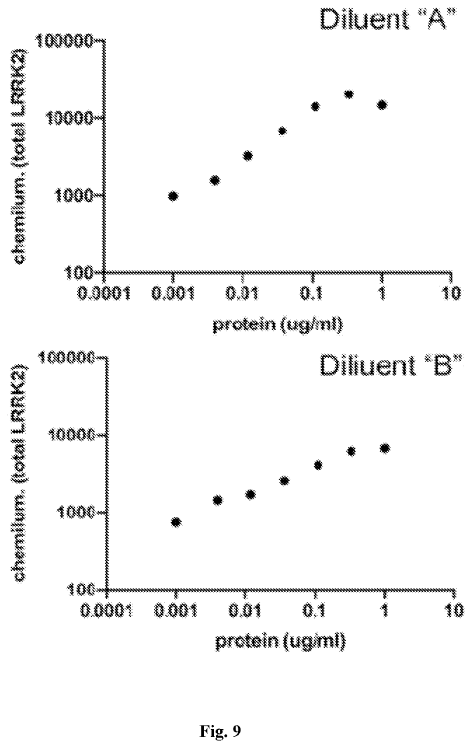

Next, over-expressed LRRK2 in HEK293T cell lines was used to compare several diluents to assess multiple dilution factors in each buffer and determine the resulting chemiluminescence signal for total LRRK2. FIG. 9 shows the results comparing two diluents in the detection of over-expressed WT human LRRK2.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method for making a clinical decision in an individual having, suspected of having, or at risk of progressing to a neurodegenerative disease or disorder, comprising:
a) performing an assay on a biological sample obtained from the individual having, suspected of having, or at risk of progressing to the neurodegenerative disease or disorder to determine a level, post-translational modification, or both of LRRK2, alpha-synuclein, Rab8a, Rab10, Rab12, and Rab29; and
b) making the clinical decision based on the level, post-translational modification, or both of LRRK2, alpha-synuclein, Rab8a, Rab10, Rab12, and Rab29.

2. The method of claim 1, wherein the clinical decision is determining at least one of a prognosis for the individual; eligibility of the individual for a clinical trial; design of a clinical trial; drug screening; dose finding; efficacy of a drug in a clinical trial; target engagement of an investigational compound; exclusion criteria, inclusion criteria, or both for a clinical trial of an investigational compound; a risk of the individual in developing the neurodegenerative disease or disorder; a stage of the neurodegenerative disease or disorder in the individual; a severity of the neurodegenerative disease or disorder in the individual; a neurodegenerative disease or disorder therapy; or an endpoint for the neurodegenerative disease or disorder therapy.

3.-15. (canceled)

16. The method of claim 1, further comprising determining an individual's responsiveness to the neurodegenerative disease or disorder therapy based on the level, post-translational modification, or both of LRRK2, alpha-synuclein, Rab8a, Rab10, Rab12, and Rab29.

17. The method of claim 16, wherein determining the individual's responsiveness to the neurodegenerative disease or disorder therapy comprises determining a likelihood of an adverse response or beneficial response to the neurodegenerative disease or disorder therapy.

18. (canceled)

19. The method of claim 1, further comprising modifying the neurodegenerative disease or disorder therapy based on the level, post-translational modification, or both of LRRK2, alpha-synuclein, Rab8a, Rab10, Rab12, and Rab29.

20. The method of claim 1, further comprising repeating steps a) to b) with a biological sample from the individual after the individual has received the neurodegenerative disease or disorder therapy and monitoring effectiveness of the neurodegenerative disease or disorder therapy by monitoring for a change in the level, post-translational modification, or both of LRRK2, alpha-synuclein, Rab8a, Rab10, Rab12, and Rab29.

21. The method of claim 1, wherein the neurodegenerative disease or disorder therapy is selected from the group consisting of a small molecule, an immunotherapy, a gene therapy, a non-medication based therapy, an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, and combinations thereof.

22. The method of claim 1, wherein the assay measures total protein, phosphorylated protein, or aggregated protein.

23. The method of claim 22, wherein the aggregated protein is selected from the group consisting of amorphous aggregates, oligomers, fibrils, and combinations thereof.

24. The method of claim 1, wherein the level is a concentration or a ratio of phosphorylation to total protein.

25. (canceled)

26. The method of claim 1, wherein the LRRK2 is at least one of total LRRK2, pS935-LRRK2, or pS1292-LRRK2.

27. (canceled)

28. (canceled)

29. The method of claim 1, wherein the alpha-synuclein is at least one of total alpha-synuclein, pS129 alpha-synuclein, or aggregated alpha-synuclein.

30. (canceled)

31. (canceled)

32. The method of claim 1, wherein the Rab8a is at least one of total Rab8a or pT72-Rab8a.

33. (canceled)

34. The method of claim 1, wherein the Rab10 is at least one of total Rab10 or pT73-Rab10.

35. (canceled)

36. The method of claim 1, wherein the Rab12 is at least one of total Rab12 or pS106-Rab12.

37. (canceled)

38. The method of claim 1, wherein the Rab29 is at least one of total Rab29 or pT71-Rab29.

39. (canceled)

40. The method of claim 1, wherein step b) comprises determining a level of at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

41. (canceled)

42. (canceled)

43. The method of claim 1, wherein the assay is an immunoassay selected from the group consisting of a Western blot, Dot blot, enzyme-linked immunosorbent assays (ELISA), chemiluminescence, absorbance, and chromatography.

44. (canceled)

45. (canceled)

46. The method of claim 43, wherein the ELISA comprises antibodies to at least two proteins selected from the group consisting of total LRRK2, pS935-LRRK2, pS1292-LRRK2, total alpha-synuclein, pS129 alpha-synuclein, aggregated alpha-synuclein, total Rab8a, pT72-Rab8a, total Rab10, pT73-Rab10, total Rab12, pS106-Rab12, total Rab29, and pT71-Rab29.

47.-51. (canceled)

52. The method of claim 1, wherein the neurodegenerative disease or disorder is selected from the group consisting of Parkinson's disease, multiple system atrophy (MSA); amyotrophic lateral sclerosis (ALS), progressive supranuclear palsy (PSP), corticobasal syndrome, Alzheimer's disease, frontotemporal dementia, multiple sclerosis (MS), corticobasal degeneration, motor neuron disease, spinocerebellar ataxia (SCA), spinal muscular atrophy (SMA), Huntington's disease (HD), Lewy body disease, Friedrich's ataxia, and synucleinopathies.

53.-55. (canceled)
